(19) United States
(12) Patent Application Publication
(10) Pub. No.: US 2006/0148103 A1
(43) Pub. Date: Jul. 6, 2006

(54) Highly sensitive biological assays

(76) Inventors: Yin-Peng Chen, Yorba Linda, CA (US); Stephan G. Thompson, El Segundo, CA (US); Thomas H.` Grove, Manhattan Beach, CA (US)

Correspondence Address: TRASK BRITT, P.O. BOX 2550, SALT LAKE CITY, UT, 84110, US

(21) Appl. No.: 11/027,422

(22) Filed: Dec. 30, 2004

(51) Int. Cl. G01N 33/551

(52) U.S. Cl. 436/524

(57) ABSTRACT

Biological assay systems with enhanced sensitivity may employ waveguides. Quantum dots, which are inorganic fluorescent markers, may be used as markers that will indicate a presence of amount of one or more target molecules (e.g., analytes) in a sample. Multiple markers may be bound to each target molecule or a corresponding competitive molecule to provide a more intense signal from each such molecule and, thus, increased sensitivity. Such multiple binding may be effected by cascading techniques, by binding markers to multiple sites on a target molecule or corresponding competitive molecule, or otherwise.

### BACKGROUND OF THE INVENTION

1. Field of the Invention

The present invention relates generally to biological assays that employ waveguides and, more specifically, to waveguide-based assays with increased sensitivity. In particular, a waveguide-based assay that incorporates teachings of the present invention may employ quantum dots to indicate whether a “target molecule,” such as an analyte or other molecule of interest, is present in a sample and, optionally, an amount of the analyte or other molecule that is present in the sample.

2. Background of Related Art

Waveguides are structures into which light may be introduced and totally internally reflected. They may be formed from organic materials, such as optical plastics, or from inorganic materials, such as glass, sapphire, and the like, that are useful for optical purposes. Their application to a variety of technologies, including optical networks, optical processors, and biological diagnostic devices, has been explored.

An exemplary biological assay system that employs a waveguide is disclosed in U.S. Pat. No. 6,738,141, the operation of which is governed by a phenomenon known as surface plasmon resonance (SPR).

While waveguides are vital to the operation of SPR type assays, they are also useful in fluorescence-based assays, as evidenced by the disclosures of U.S. Pat. Nos. 5,512,492, 5,846,842, 5,677,196, 6,108,463, 6,222,619, 6,242,267, 6,287,871, 6,316,274, and 6,611,634, the disclosures of each of which are hereby incorporated herein, in their entireties, by this reference. Particularly, fluorescence-based assays that employ waveguides have been found to provide fairly accurate results in short periods of time, even with very small sample volumes.

When used in a biological assay, capture molecules are typically immobilized on a surface of the waveguide. The assay process typically includes exposure of the surface and, thus, the capture molecules to a sample that may include one or more analytes or other molecules of interest. In addition, the capture molecules or the sample are exposed to one or more reagent solutions, which include indicators or markers, such as fluorescent dyes, metal particles, or the like. Electromagnetic radiation of at least one wavelength that will excite the indicators or markers is introduced into the waveguide (e.g., through an edge of a planar waveguide) and is internally reflected within the waveguide. Such internal reflection results in the generation a phenomenon known as an “evanescent field” over the major surfaces of the waveguide. Although the evanescent field extends a predetermined distance over each major surface, the capture molecules and any molecules that are bound thereto are present within the evanescent field. Due to the limited extent of the evanescent field, it does not encompass the vast majority of unbound molecules, including most of the unbound indicators or markers. The evanescent field will only affect the indicator or marker molecules that are present therein. For example, fluorescent indicators or markers will fluoresce, or emit light, when exposed to an evanescent field generated by internal reflection of an appropriate wavelength of electromagnetic within the waveguide. As another example, metallic indicators or markers will oscillate when exposed to an evanescent field generated by internal reflection of an appropriate wavelength of electromagnetic within the waveguide. The affects of the evanescent field on the indicators or markers may be detected, either by evaluating electromagnetic radiation that exits the waveguide (e.g., through an edge of a planar waveguide), or by more directly evaluating the affects of the evanescent field on the indicators or markers (e.g., by orienting a detector toward the surface of the waveguide (either directly or indirectly). As the vast majority of indicator or marker molecules that are present within the evanescent field are indirectly immobilized relative to the surface of the waveguide, evaluation of the affects of the evanescent field on the indicators or markers may provide an accurate and reliable indication of the amount of an analyte or other molecule of interest that is present in the sample.

Conventionally, organic fluorescent molecules, or dyes, which are also known in the art as “organic fluorophores,” have been used in fluorescent waveguide assays. The use of fluorescent dyes is somewhat undesirable, however, in that they may not provide the desired degree of sensitivity (intensity of light emitted per molecule, collective intensity, etc.). Moreover, fluorescent dyes are often excited by a relatively narrow range of wavelengths of electromagnetic radiation, which may limit the types of fluorescent dyes that may be used with a particular device or, conversely, increase the cost of a device by requiring multiple sources of electromagnetic radiation or multiple optical filters. In addition, fluorescent dyes typically emit electromagnetic radiation of a relatively broad range of wavelengths, which may decrease the ability to distinguish emissions from different types of dyes. Furthermore, the difference, in nanometers, between the peak excitation and emission wavelengths of organic fluorescent molecules, or “Stoke's shift,” is typically relatively small.

Each of these attributes of organic fluorescent molecules, or dyes, may contribute to the typically undesirably low sensitivity of fluorescent waveguide assays.

Accordingly, there are needs for waveguide-based assays that will detect analytes or other molecules of interest with improved sensitivity.

### SUMMARY OF THE INVENTION

The present invention includes biological assay systems with increased sensitivity and techniques that are believed to increase the sensitivity of such biological assay systems.

Biological assay systems that incorporate teachings of the present invention may comprise sandwich assays, competitive binding, or “competition,” assays (see, e.g., U.S. Pat. Nos. 6,482,655 and 6,632,613, the disclosures of both of which are hereby incorporated herein in their entireties, by this reference) or any other known assay type. These assay systems may include optical waveguides or other types of substrates.

An exemplary embodiment of assay system according to the present invention includes a waveguide, at least one type of capture molecule carried upon at least one surface of the waveguide, and a reagent solution. The capture molecule selectively binds a complementary species of target molecule present within a sample (in a sandwich assay) or a corresponding competitive molecule (in a competition assay). In a sandwich assay, the reagent solution includes a signal complex with a marker that, when bound to the target molecule or corresponding competitive molecule, provides an indication of the presence of the target molecule in the sample and, optionally, of an amount of the target molecule present in the sample. When a competition assay is being conducted, the reagent solution includes the corresponding competitive molecule, which may be directly or indirectly labeled with the marker. By way of example only, the marker may comprise a so-called “quantum dot,” which is an inorganic fluorescent molecule.

Such an assay system may also include an excitation source, which is oriented to direct, into the waveguide, excitation radiation of a wavelength that will excite any markers that are present within an evanescent field generated at a surface of the waveguide-primarily those markers that have been immobilized relative to the surface of the waveguide. When quantum dots or other fluorescent markers are used, excitation thereof results in the fluorescence of emission radiation. Such emission radiation may be detected by an optical detector element, or “detector,” of the assay system.

In another aspect, the present invention includes a method for effecting a biological assay. An example of such a method includes exposing a sample solution that potentially includes at least one species of target molecule to a reagent solution. For sandwich assays, the reagent solution may include at least one type of signal complex that is configured to bind directly or indirectly to target molecules and, thus, to secure markers to the target molecules. For competition assays, the reagent solution includes competitive molecules that are labeled directly or indirectly with markers (e.g., quantum dots). In either event, the markers indicate a presence or an amount of the at least one target molecule in the sample.

The sample solution is introduced onto the surface of a waveguide, to which capture molecules have been immobilized, to selectively bind target molecules or corresponding competitive molecules either directly or indirectly to the capture molecules. Binding of target molecules or corresponding competitive molecules is detected by directing excitation radiation into the waveguide and detecting emission radiation that is fluoresced by markers that were present in an evanescent field generated at a surface of the waveguide. The amount of emission radiation that is detected may be correlated to an amount of the target molecule present in the sample.

According to another aspect, the present invention includes techniques for enhancing the sensitivity of an assay. For example, multiple markers may be secured to each target molecule or corresponding competitive molecule. In one such technique, markers are secured to target molecules or competitive molecules. Additional markers are then secured, relative to previously secured markers, in a cascade type arrangement.

Other features and advantages of the present invention will become apparent to those of ordinary skill in the art through consideration of the ensuing description, the accompanying drawings, and the appended claims.

### BRIEF DESCRIPTION OF THE DRAWINGS

In the drawings, which depict exemplary embodiments of various aspects of the present invention:

FIG. 1 is a schematic representation of an exemplary embodiment of biological assay system, which includes a waveguide, according to the present invention;

FIG. 2 schematically depicts indirectly immobilization of target molecules to a solid phase, in this case oligonucleotides to capture nucleic acid molecules on a waveguide surface;

FIGS. 3A through 3C schematically depict a first exemplary technique for labeling a target molecule (e.g., a nucleic acid) with markers, which are conjugated to binding pair members (e.g., signal oligonucleotides) that directly hybridize with complementary regions (i.e., the other member of the binding pair) (e.g., nucleotide sequences) of the target molecule;

FIG. 4 schematically depicts a variation of the first exemplary technique, in which different locations (e.g., nucleotide sequences) of each target molecule may be labeled with multiple markers;

FIG. 5 schematically depicts a second exemplary technique for labeling a target molecule (e.g., a nucleic acid), in which markers are conjugated with binding pair members (e.g., signal oligonucleotides) that hybridize with probe molecules which, in turn, hybridize with complementary regions (e.g., nucleotide sequences) of the target molecule; thus, the binding pair member that has been conjugated with the marker is indirectly secured to the target molecule;

FIG. 6 is a schematic representation of second exemplary technique for labeling a target molecule, in which one member of a binding pair is conjugated to regions of a the target molecule, while the other member of the binding pair is conjugated to the marker with which the target molecule is to be labeled;

FIG. 7 is a schematic representation of a variation of the labeling technique shown in FIG. 6, in which one member of a binding pair is conjugated to an intermediate probe molecule (e.g., a probe oligonucleotide) that, in turn, binds to or hybridizes with a complementary region (e.g., a nucleotide sequence) of a target molecule (e.g., a nucleic acid);

FIG. 8 schematically illustrates amplification of a marker signal by labeling a target molecule, then cascading additional marker molecules onto marker molecules that have already been bound to the target molecule;

FIGS. 9A through 9C are photographs showing increasing intensities with no cascading (see, e.g., FIG. 7), a first degree of cascading (see, e.g., FIG. 8), and a second degree of cascading (see, e.g., FIG. 8), respectively; and

FIG. 10 is a schematic representation of an exemplary immunoassay according to the present invention.

### DETAILED DESCRIPTION

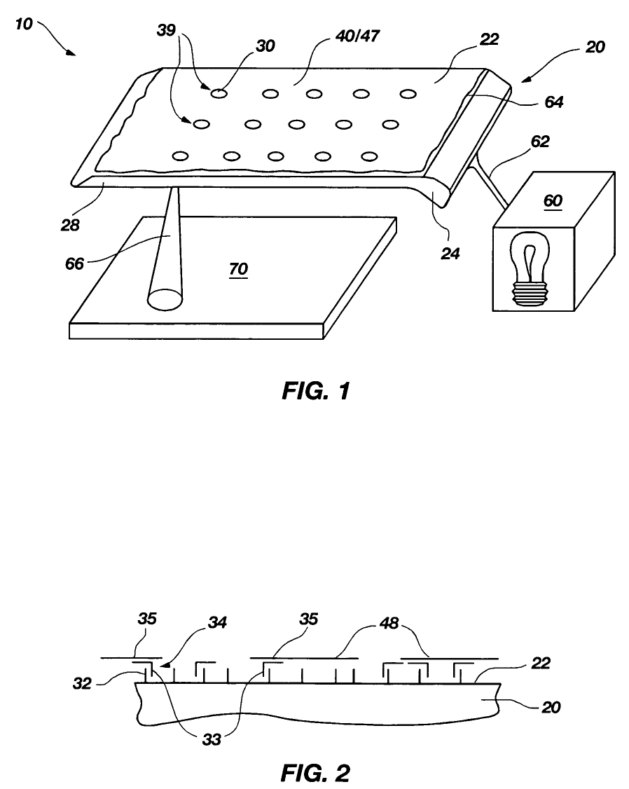

FIG. 1 depicts an exemplary assay system 10 that incorporates teachings of the present invention. Assay system 10 includes a waveguide 20, a source 60 of electromagnetic radiation, and a detector 70. Capture molecules 30 that have binding specificity for at least one species of analyte or other molecule of interest (i.e., a particular analyte, such as a specific nucleic acid sequence, an antibody with a particular antigen-specificity, an antigen with a particular epitope, etc.) are secured, or “immobilized,” to a surface 22 of waveguide 20. Assay system 10 may also include a reagent solution 40. Reagent solution 40, which is used when capture molecules 30 are exposed to a sample 47, facilitates a determination of whether or not the analyte or other molecule of interest is present in the sample, or quantification of the amount of the analyte or other molecule of interest in the sample.

Although waveguide 10 is shown in FIG. 1 as a planar waveguide which includes a ramped lens 24 at one end, any other suitable waveguide configuration may be used in accordance with teachings of the present invention. For example, and not by way of limitation, waveguide 10 may comprise a completely planar waveguide, a cylindrical waveguide, a waveguide having an elongate prismatic shape, a spherical waveguide, or the like.

Waveguide 10 may be formed from any suitable material. Exemplary waveguide materials include both organic materials (i.e., optical plastics, such as polycarbonates, polystyrenes, polyvinylchloride (PVC), etc.) and inorganic materials (e.g., glass (e.g., borosilicate glass), silicon dioxide, silicon oxynitride, sapphire, etc.). Such materials may form the portion of waveguide 10 within which light is internally reflected.

Alternatively, a so-called “thin film waveguide” may include a thin film carried upon a surface of a substrate. The substrate may be formed from any suitable optical material, including, but not limited to, those described in the preceding paragraph, that will transmit electromagnetic radiation (e.g., light) into the thin film. The thin film, in which light is internally reflected, is formed from a material that has a higher refractive index than that of the material from which the substrate is formed. Exemplary materials that may be used as the thin film include, without limitation, Ta₂O₅, TiO₂, and the like. By way of nonlimiting example, the thin film may have a thickness of about 150 nm to about 300 nm.

Capture molecules 30 may be immobilized to surface 22 of waveguide 20 in any suitable fashion, with a variety of techniques being known in the art. For example, capture molecules 30 may be secured to surface 22 by opposite electrostatic charge, by crosslinking (e.g., with an ultraviolet (UV) crosslinker, such as one of the Stratalinker® UV crosslinkers available from Stratagene Corporation of La Jolla, Calif., and described in U.S. Pat. Nos. 5,288,647 and 5,395,591, the disclosures of both of which are hereby incorporated herein, in their entireties, by this reference, or the heterobiofunctional crosslinkers that are described in U.S. Pat. Nos. 5,279,955 and 5,436,417, the disclosures of both of which are hereby incorporated herein, in their entireties, by this reference), or by any other suitable means for immobilization.

As illustrated, capture molecules 30 may be arranged as an array of spots 39. Of course, any other arrangement of capture molecules 30 on surface 22 is also within the scope of the present invention. For example, capture molecules 30 may cover, in substantially confluent fashion, a larger portion or all of surface 22. As another example, capture molecules 30 may be arranged in one or more strips on surface 22.

Further, surface 22 of waveguide 20 may be coated capture molecules 30 that bind specifically to a single species of analyte or other molecule of interest, or with capture molecules 30 of different specificities. These different species of capture molecules 30 may remain separate from one another and, thus, spaced at different locations on surface 22 or they may be randomly mixed with one another on surface 22.

Reagent solution 40 includes one or more components that are configured to result in the detection of the presence or amount of an analyte or other molecule of interest, which are collectively referred to herein as “target molecules,” in a sample 47. Among other components, various examples of which are provided in the EXAMPLES that follow, a reagent solution 40 that incorporates teachings of the present invention includes a marker. Without limiting the scope of the present invention, the marker may comprise an inorganic fluorescent molecule (e.g., a quantum dot), an organic fluorescent molecule (e.g., fluorescein, CY-5 dye, CY-7 dye, etc.), a metallic particle, an enzymatic marker (e.g., horseradish peroxidase), or any other type of marker suitable for use in biological waveguide assays.

“Quantum dots” are crystals that have dimensions that may be measured in nanometers (nm) (e.g., from about 2 nm to about 1,000 nm across) and, thus, may also be referred to as “nanocrystals.” They are formed from semiconductor materials, such as elements that make up groups II through IV, III through V, or IV through VI of the Periodic Table of the Elements. For example, quantum dots may include cadmium (Cd)/selenium (Se) cores and zinc sulfide (ZnS) shells and, thus, be identified by the chemical formula CdSeZnS.

Different types of quantum dots are excited when exposed to different ranges of wavelengths of electromagnetic radiation. Currently available quantum dots may be excited by electromagnetic radiation having wavelengths as low as about 300 nm and as high as about 2,300 nm.

The optical properties of quantum dots are primarily dictated by their physical size and chemistry. Typically, electromagnetic radiation having a wavelength within the visible light and infrared portions of the spectrum will excite quantum dots. The absorption spectrum of a quantum dot appears as a series of overlapping peaks that become increasingly larger at decreasingly shorter wavelengths. Each peak corresponds to an energy transition between discrete electron-hole energy states (exciton) within the quantum dot. The size of a quantum dot and the difference between its energy states are inversely proportional. Thus, the difference between energy states of larger quantum dots is smaller than the difference between energy states of smaller quantum dots.

The smaller the difference between the energy states of a quantum dot, the “redder” (or higher wavelength) of the electromagnetic radiation (e.g., light) emitted therefrom. Thus, when excited, larger quantum dots will emit “redder” light than smaller quantum dots, which will emit “bluer” light. As a consequence of these phenomena, the wavelength of electromagnetic radiation emitted by a quantum dot may be tailored by selecting the material from which the quantum dot is to be synthesized and the size to which the quantum dot is to be synthesized. When excited, known quantum dots may emit electromagnetic radiation (e.g., light) having a wavelength from about 490 nm (blue) to about 705 nm (red).

Quantum dots have high quantum yields and resist photobleaching; their use therefore providing for very sensitive fluorescent biological assays.

It is currently preferred that the markers within reagent solution have a Stoke's shift of about 50 nm or greater (e.g., the difference between excitation of the marker at about 658 nm and emission at about 703 nm) or even of about 100 nm or greater (e.g., quantum dots that are excited at about 405 nm may emit radiation having a wavelength of about 530 nm).

The following EXAMPLE provides details on the manner in which a member of a binding pair may be conjugated to a quantum dot.

### EXAMPLE 1

Fort orange amine-EVITAGS 600 nm quantum dots from Evident Technologies, Inc., of Troy, Mich., which are “ready for conjugation,” were conjugated with biotin molecules. A 400 picomole (pmole) aliquot of the quantum dots was buffer-exchanged into 0.1M sodium borate, pH 8.3, with an AMICON® CENTRICON® YM-30 centrifugation filter device, which is available from Millipore Corporation of Billerica, Mass. The quantum dots were then biotinylated, for one hour at 37° C., in 0.50 mL of borate buffer with a sixty-fold excess of EZ-LINK® NHS-LC-LC-Biotin, which is available from Pierce Biotechnology, Inc., of Rockford, Ill. The biotinylation reaction was quenched with 12.5 μmole Tris-HCl, pH 8.1, for 15 minutes. The biotinylated quantum dots were then desalted, as known in the art, on a CENTRICON® YM-30 centrifugation filter device. Following desalting, the quantum dots were resuspended in 200 μL of borate buffer with 00.05% Tween-20.

With continued reference to FIG. 1, source 60 may comprise any suitable source of electromagnetic radiation. As an example, source may comprise a laser. More specifically, source 60 may comprise a laser that emits a beam 62 of electromagnetic radiation having a wavelength (e.g., 405 nm, 658 nm, etc.) that will excite one or more species of marker within reagent solution 40.

Detector 70 is configured to sense radiation of one or more wavelengths emitted from the quantum dots of the reagent solution. By way of example, without limiting the scope of the present invention, detector may be configured to sense electromagnetic radiation having wavelengths of about 600 nm to about 650 nm. Exemplary devices that may be employed as detector 70 include, but are not limited to, charge-coupled displays (CCD), complementary metal-oxide-semiconductor (CMOS) imager, photodiodes and the like.

Turning now to FIG. 2, an exemplary nucleic acid assay that incorporates teachings of the present invention is schematically depicted. In FIG. 2, a surface 22 of a waveguide 20 is shown.

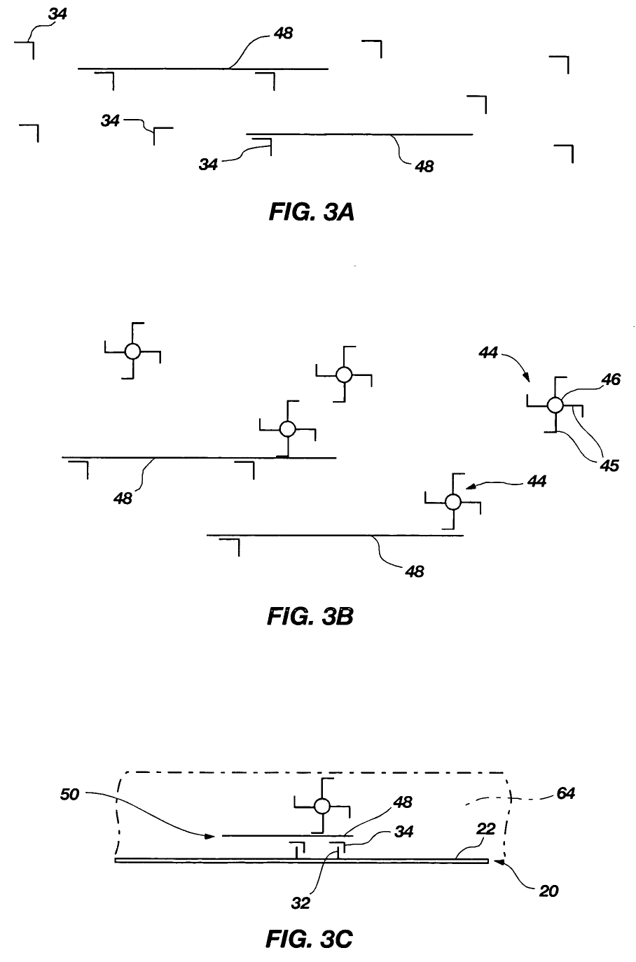

Capture oligonucleotides 32 are immobilized to surface 22 by known processes (e.g., electrostatic attraction, crosslinkers, etc.). Capture oligonuclotides 32 may have a sequence of nucleotides that will hybridize with unique, complementary nucleotide sequence on a target molecule. Alternatively, as shown, capture oligonucleotides 32 may have a relatively unique sequence of nucleotides that facilitates hybridization with a complementary capture oligonucleotide-specific region 33 of a bridge oligonucleotide 34, which is part of a reagent solution 40 (FIG. 1) while minimizing hybridization with other single-stranded nucleic acid sequences, such as those of the target molecules 48 and probe oligonucleotides (shown as 42a, 42b, 42c, etc., in FIGS. 5A through 5C) or signal oligonucleotides (shown as signal oligonucleotides 45 in FIGS. 3A through 3C and as signal olignucleotides 45′ in FIG. 5).

In addition to capture oligonucleotide-specific region 33, each bridge oligonucleotide 34 includes a target molecule-specific region 35, which has a sequence of nucleotides that is complementary to a nucleotide sequence of a particular region of target molecule 48. Thus, it is the target molecule-specific region 35 of each bridge oligonucleotide that is responsible for hybridizing to and, thus, immobilizing, a target molecule 48.

Since capture oligonucleotides 32 are used to hybridize with a unique sequence on a bridge oligonucleotide 34 which, in turn, hybridizes with a region of a target molecule 48 to immobilize the same to surface 22 of waveguide 20, they are not specific to target molecule 48. Accordingly, these capture oligonucleotides 32 may be used with bridge oligonucleotides 34 that have a variety of different target molecule-specific regions 35.

As a result of such nonspecificity, capture oligonucleotides 32 may hybridize with bridge oligonucleotides 34 that bind to different regions of a particular target molecule 48. Thus, the likelihood that target molecule 48 will be immobilized to surface 22 and subsequently detected may be increased, which may result in an increase in the overall sensitivity of the assay.

Moreover, a waveguide 20 that has such universal capture oligonucleotides 32 immobilized to surface 22 thereof may be used in a single assay for two or more target molecules 48. Two or more assays may be concurrently effected by merely providing bridge oligonucleotides 34 with capture oligonucleotide-specific regions 33 that have sequences that will hybridize with the sequence of capture oligonucleotides 32 and target molecule-specific regions 35 that have sequences that are complementary to and, thus, will hybridize with particular regions of two or more particular species of target molecule 48.

### EXAMPLE 2

A BioCentrex cartridge, available from BioCentrex, LLC, of Culver City, Calif., that included a planar waveguide with a surface that had been spotted with 0.5 μM NeutrAvidin®, available from Pierce Biotechnology, Inc., of Rockford, Ill., was used to evaluate binding between the members of a binding pair-namely, biotin and NeutrAvidin®.

QDOT® 655 Biotin Conjugate biotinylated quantum dots, obtained from Quantum Dot Corporation of Hayward, Calif., were suspended in 50 mM sodium borate, pH 8.3, at concentrations of 0 nM, 1 nM, 5 nM, and 10 nM. A 300 μL sample of each solution was placed in the reagent cup of a BioCentrex cartridge. The cartridge was then inserted into a BioCentrex analyzer, which had been equipped with a 658 nm red laser, a 703 nm band pass filter, and a CCD camera.

As the 658 nm red laser introduced a laser beam into an edge of the waveguide, the binding reaction between the biotinylated quantum dots and the NeutrAvidin® spots was monitored with the CCD camera. Such monitoring was effected for a duration of eight minutes, with two second exposure times that were spaced at 6.5 second intervals. The average fluorescent rates, which is a measure of the change in fluorescent intensity per minute (e.g., with a CCD camera, CMOS imager, or photodiode), are presented in TABLE 1.

TABLE 1

| Concentration of | Average |
|---|---|
| QDOT ®-Biotin Conjugate | Fluorescent Rate |
| 0 nM | 0 |
| 1 nM | 4.2 |
| 5 nM | 19.6 |
| 10 nM | 41.8 |

The average fluorescent rates are directly proportionate to the concentration of biotinylated quantum dots that contacted with the NeutrAvidin® spots on the surface of the waveguide.

### EXAMPLE 3

A BioCentrex Analyzer that had been equipped with a 405 nm blue-violet laser, a 530 nm long pass filter, and a CCD camera was used to evaluate the average fluorescent rates generated when such a laser was used to excite the EviTag® biotinylated quantum dots described in EXAMPLE 1 and QDOT® 655 Biotin Conjugate biotinylated quantum dots.

A 1.0 nM solution of the EviTag® biotinylated quantum dots, a 1.0 nM solution of the QDOT® 655 Biotin Conjugate biotinylated quantum dots, and a 10.0 nM solution of the QDOT® 655 Biotin Conjugate biotinylated quantum dots, each suspended in 50 mM sodium borate, pH 8.3, were evaluated. A 300 μL sample of each quantum dot solution was introduced into the reagent cup of a separate BioCentrex cartridge, then introduced onto a NeutrAvidin®-spotted surface of a planar waveguide of the cartridge. The cartridge was then introduced into the BioCentrex analyzer.

As the 405 nm blue-violet laser introduced a laser beam into an edge of the waveguide, the binding reaction between the biotinylated quantum dots and the NeutrAvidin® spots was monitored with the CCD camera. Such monitoring was effected for a duration of eight minutes, with 0.2 second exposure times that were spaced at 6.5 second intervals. The average fluorescent rates are presented in TABLE 2.

TABLE 2

| Quantum Dot-Biotin | Concentration of | Average |
|---|---|---|
| Conjugate | Conjugate | Fluorescent Rate |
| EviTag ® 600 | 1.0 nM | 10.0 |
| QDOT ® 655 | 1.0 nM | 13.8 |
| QDOT ® 655 | 10.0 nM | 148.5 |

These results again show that the fluorescent rates are directly proportionate to the concentration of biotinylated quantum dots that contacted with the NeutrAvidin® spots on the surface of the waveguide.

In the EXAMPLES that follow, descriptions of various techniques that may be used to facilitate detection of the immobilization of a target molecule relative to a surface of a waveguide are provided.

EXAMPLES 4 through 6 include systems in which markers are secured to oligonucleotides.

### EXAMPLE 4

An exemplary approach for detecting whether or not at least one particular species of target molecule 48 is present in a sample, or for detecting the amount of that particular species of target molecule 48 in a sample, is shown in FIGS. 3A through 3C.

Using a sandwich assay, such as that depicted in FIG. 2, anthracis DNA was specifically and sensitively detected.

In the example, the sample included anthracis DNA that was amplified using well-known polymerase chain reaction (PCR).

With reference to FIG. 3A, the sample, which included target molecules 48, was exposed to bridge oligonucleotides 34, which may be part of a reagent solution 40 (FIG. 1). More specifically, the PCR-amplified anthracis DNA was mixed with a 20 nM concentration of bridge oligonucleotide 34. The mixture was subjected to an increased temperature, or “heat denatured,” as known in the art, to facilitate separation of the two strands of the anthracis DNA and, thus, to permit the bridge probe to bind to complementary locations, or sites, along the lengths of the single strands of the anthracis DNA. As time progressed and this mixture was incubated, bridge oligonucleotides 34 hybridized with complementary portions of target molecules 48. Bridge oligonucleotides 34 facilitate hybridization between capture oligonucleotides 32 on surface 22 of waveguide 20 and a target molecule 48-in this case, anthracis DNA.

Next, the anthracis DNA-bridge oligonucleotide complexes were exposed to a reagent solution that included a 2 nM concentration of signal oligonucleotide-quantum dot complexes to form a sample-reagent mixture and to permit the signal oligonucleotides of the signal oligonucleotide-quantum dot complexes to hybridize to complementary regions of the anthracis DNA and, thus, to form a probe-analyte-bridge complex.

An example of the introduction of a reagent solution 40 (FIG. 1) into the presence of a sample that may include, among other things, target molecules 48 is illustrated in FIG. 3B. As shown, reagent solution 40 includes signal complexes 44. Each signal complex 44 includes a marker 46 and one or more signal oligonucleotides 45 secured to marker 46. Each signal oligonucleotide 45 includes a nucleotide sequence that will hybridize with a complementary nucleotide sequence along at least a portion of target molecule 48. As used in testing, signal oligonucleotide 45 of each signal complex 44 included a nucleotide sequence complementary to nucleotide sequences substantially unique to anthracis DNA (e.g., SP6 oligonucleotides). Markers 46, to which signal oligonucleotides 45 are secured by known techniques, may comprise quantum dots, organic fluorescent dye molecules, or the like. In the signal complexes 44 that were used in the tests, markers 46 were quantum dots.

When the sample was exposed to, or incubated with, signal complexes 44, signal oligonucleotides 45 hybridized with complementary regions of any target molecules 48 in the sample, effectively securing markers 46 to target molecules 48.

Thereafter, the sample-reagent mixture was introduced onto a capture molecule-bearing surface of the waveguide. This is shown in FIG. 3C. Upon exposure of bridge oligonucleotide 34 of the signal-analyte-bridge complex 50 and capture oligonucleotides 32 on surface 22 of waveguide 20 to one another, complementary sequences of both hybridized to each other, which immobilized at least some target molecules 48 (i.e., the anthracis DNA) to the capture oligonucleotide-bearing surface 22 of waveguide 20.

As illustrated in FIG. 1, such immobilization was detected by introducing excitation radiation 62 (in this case, near-ultraviolet radiation) into waveguide 20 (in this case, into an edge, or ramped lens 24, of the illustrated planar waveguide 20) with a source of electromagnetic radiation (in this case, a violet (405 nm) laser) (not shown). Internal reflection of excitation radiation 62 within waveguide 20 resulted in the generation of an evanescent field 64 at surface 22 of waveguide 20. Any marker 46 (FIG. 3C) within evanescent field 64 (primarily markers 46 that were immobilized relative to target molecule 48 (FIG. 3C) and surface 22) was excited and, thus, fluoresced emission radiation 66. Such evanescent field-generated emission radiation 66 was detected with a detector 70 oriented transversely to a plane in which the capture molecule-bearing surface was located (in this case, a CCD camera oriented toward a surface 28 of waveguide 20 which is opposite from surface 22), as known in the art.

### EXAMPLE 5

One of the ways to increase the sensitivity with which a molecule of interest (e.g., a nucleic acid, a protein, etc.) is detected includes increasing the number of markers (e.g., quantum dots, organic fluorescent markers, metal particles, etc.) that attach to the molecule of interest, or the marker-to-target molecule ratio.

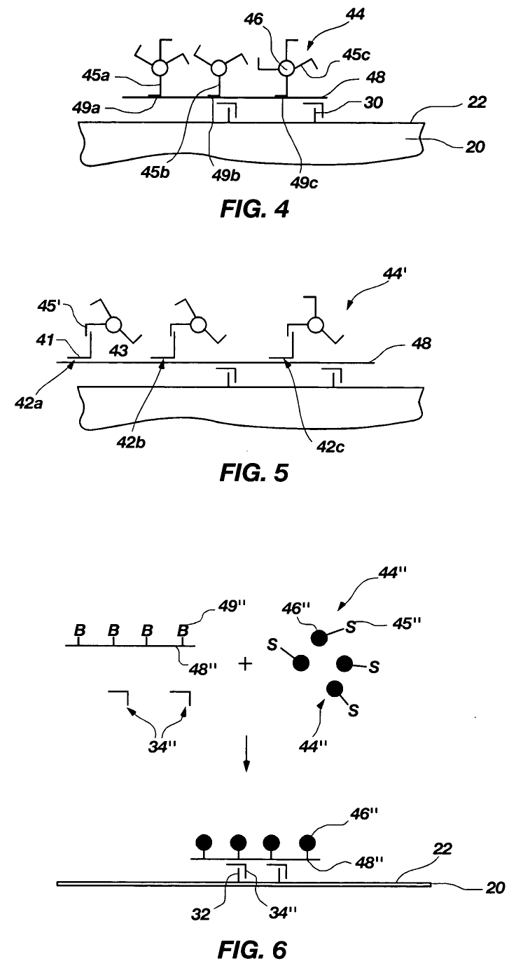

A first exemplary approach to increasing the marker-to-target molecule ratio is schematically illustrated in FIG. 4. In this approach, when the target molecule 48 is a nucleic acid, reagent solution 40 (FIG. 1) includes marker-labeled signal oligonucleotides 45a, 45b, 45c, etc., that are complementary to a respective plurality of different sites 49a, 49b, 49c, etc., (e.g., unique sequences) of target molecule 48. Continuing with the previous example in which anthracis DNA was the target molecule 48, signal oligonucleotides 45a, 45b, 45c, etc., may include SP1, SP6, and SP7 oligonucleotides, which hybridize with different complementary sites on a strand of anthracis DNA.

Thus, more than one marker-labeled signal oligonucleotide 45a, 45b, 45c, etc., can hybridize with or otherwise bind to each target molecule 48 and, as a consequence, a corresponding number of markers 46 (e.g., quantum dots, organic fluorescent dye molecules, etc.) are immobilized relative to each target molecule 48. If target molecule 48 has hybridized with or otherwise been bound by one or more complementary capture molecules 32, markers 46 are also immobilized near surface 22 of waveguide 20 and, therefore, are likely to be exposed to an evanescent field generated at surface 22. As a result, an increased number of markers 46 will be excited by the evanescent field, increasing the intensity of a signal (e.g., fluorescent radiation in the case of quantum dots and organic fluorescent markers) that is emitted per immobilized, or “captured,” target molecule 48.

When a plurality of different species of target molecules 48 are being assayed using the same waveguide 20, it may be necessary to distinguish between the different species of assayed target molecules 48 that are present in a sample. Such distinctions may be made by using signal complexes 44 with distinctive markers 46, each of which corresponds to a particular species of target molecule 48 (e.g., by generating distinctive signals when excited). Each species of signal complex 44 may include one or more signal oligonucleotides 45 with a sequence of nucleotides that is configured to hybridize with a complementary sequence of nucleotides along a region of a particular species of target molecule 48, as well as a marker 46 that provides a distinctive signal that corresponds to that particular species of target molecule 48. For example, when fluorescent molecules, such as quantum dots or organic fluorescent dyes, are used as markers 46 of signal complexes 44, markers 46 that, when excited, fluoresce emission radiation 66 of distinctive wavelengths may be used to facilitate a distinction between the presence or absence or amounts of each of the assayed species of target molecule 48 present in the sample.

### EXAMPLE 6

Another embodiment of the present invention, illustrated in FIG. 5, includes signal complexes 44′ that are not specific for a particular species of target molecule 48, as are signal complexes 44 (FIGS. 3A through 3C and 4) that include signal oligonucleotides 45 configured to hybridize directly with and, thus, “label” a target molecule with markers 46. Instead, signal complexes 44′ are indirectly bound to and, thus, indirectly label target molecules 48.

More specifically, as shown in FIG. 5, signal complexes 44′ are configured to be used in conjunction with probe oligonucleotides 42a, 42b, 42c, etc., (which are also collectively referred to herein as “probe oligonucleotides 42”).

Each probe oligonucleotide 42 includes a target molecule-specific region 41 and a signal oligonucleotide-specific region 43. Target molecule-specific region 41 has a nucleotide sequence that is configured to hybridize with a complementary sequence of nucleotides along at least a region of target molecule 48. Signal oligonucleotide-specific region 43 has a nucleotide sequence that will hybridize with a complementary sequence of nucleotides of a corresponding signal oligonucleotide 45′ of a signal complex 44′.

Different species of probe oligonucleotides 42 and signal complexes 44′ may be used concurrently to assay a plurality of different species of target molecules 48 with the same waveguide 20. Each species of probe oligonucleotide 42 includes a target molecule-specific region 41 that will hybridize with a complementary nucleotide sequence of one assayed species of target molecule 48. That species of probe oligonucleotide 42 also includes a signal oligonucleotide-specific region 43 with a sequence that will hybridize only with a signal oligonucleotide 45′ of a species of signal complex 44′ that corresponds to one assayed species of target molecule 48. The signal that is provided by marker 46 of that species of signal complex 44′ is, of course, distinguishable from the signals provided by markers of other species of signal complexes. Gene-specific bridge probes and capture probes may be used in a similar fashion to concurrently assay a plurality of different species of target molecules.

Other types of binding pairs, or ligand-receptor systems, such as biotin-biotin binding protein type systems and polyT-polyA complexes, may also be used to facilitate detection of a presence or an amount of one or more target molecules in a sample. In the following EXAMPLES, several assays that include ligand-receptor-based systems for marking target molecules are described.

### EXAMPLE 7

FIG. 6 shows an assay system in which target molecules 48″ are amplified and, during amplification, biotinylated to facilitate binding of signal complexes 44″ that comprise biotin binding protein-labeled markers thereto.

First binding pair members, such as biotin molecules 49″ or biotin binding proteins, may be incorporated into nucleic acid molecules. For example, biotin molecules 49″ may be incorporated into synthesized target molecules 48″ by including a biotin-dNTP (deoxy-[nucleotide]-triphosphate), where N represents any nucleotide (e.g., C, G, A, T, etc.), among the nucleotides that are used to amplify a nucleic acid molecule of interest (e.g., by PCR or other amplification or transcription-like activities), as is well known in the art. As a specific but nonlimiting example, anthracis DNA may be amplified by PCR using biotin-14-dCTP. The result is double-stranded biotinylated target molecules.

Each signal complex 44″ includes a marker 46″ with one or more second binding pair members, such as biotin binding proteins 45″ or biotin molecules, conjugated thereto. By way of example only, marker 46″ may comprise a quantum dot, although other types of markers (e.g., fluorescent, radioactive, metallic, enzymatic, etc.) are also within the scope of the present invention. Biotin binding protein 45″ may comprise any known type of biotin binding protein, such as avidin, streptavidin, NeutrAvidin™ (available from Pierce Biotechnology, Inc., of Rockford, Ill.), CaptAvidin™ (available from Molecular Probes, of Eugene, Oreg.), or the like. Exemplary quantum dot-biotin binding protein conjugates that may be used as signal complexes 44″ include, without limitation, one of the QDOT® Streptavidin Conjugates available from Quantum Dot Corporation of Hayward, Calif. (e.g., QDOT® 525 Streptavidin Conjugate, QDOT® 565 Streptavidin Conjugate, QDOT® 585 Streptavidin Conjugate, QDOT® 605 Streptavidin Conjugate, QDOT® 655 Streptavidin Conjugate, QDOT® 705 Streptavidin Conjugate).

After the double-stranded biotinylated target molecules have been synthesized, they may be heat denatured, which separates the two single stranded biotinylated target molecules 48″, and mixed with complementary bridge oligonucleotides 34″ (e.g., anthracis-specific bridge oligonucleotides) and with markers 46″.

When target molecules 48″ are exposed to signal complexes 44″ (e.g., during incubation), the biotin binding protein or proteins 45″ of some of the signal complexes 44″ bind to biotin molecules 49″ of target molecule 48″. As each target molecule 48″ may include multiple biotin molecules 49″, multiple signal complexes 44″ may be bound, by a biotin binding protein 45″ thereof, to that target molecule 48″. The number of signal complexes 44″ that are bound to target molecules 48″ in a sample corresponds to the collective signal intensity that may be generated by signal complexes 44″.

In addition, as target molecules 48″ are exposed to (e.g., incubated with) complementary bridge oligonucleotides 34″, complementary nucleotide sequences of bridge oligonucleotides 34″ and target molecules 48″ hybridize with one another.

As bridge oligonucleotides 34″ are exposed to capture oligonucleotides 32 that have been immobilized to a surface 22 of a waveguide 20, complementary nucleotide sequences of bridge oligonucleotides 34″ and capture oligonucleotides 32 hybridize, thereby indirectly immobilizing target molecules 48″ and any signal complexes 44″ bound thereto to surface 22.

Upon appropriate excitation (e.g., with laser light directed into waveguide 20), markers 46″ located within a given distance of surface 22 (e.g., markers 46″ that are indirectly secured to target molecules 48″ that have been immobilized relative to surface 22) are excited (e.g., by an evanescent field 64 (FIG. 1) generated at surface 22). In the example where markers 46″ comprise quantum dots or other fluorescent molecules, emission radiation 66 (FIG. 1) is emitted, providing a detectable visible light signal that corresponds to the presence of or even an amount of target molecule 48″ (e.g., anthracis DNA) present in the sample.

### EXAMPLE 8

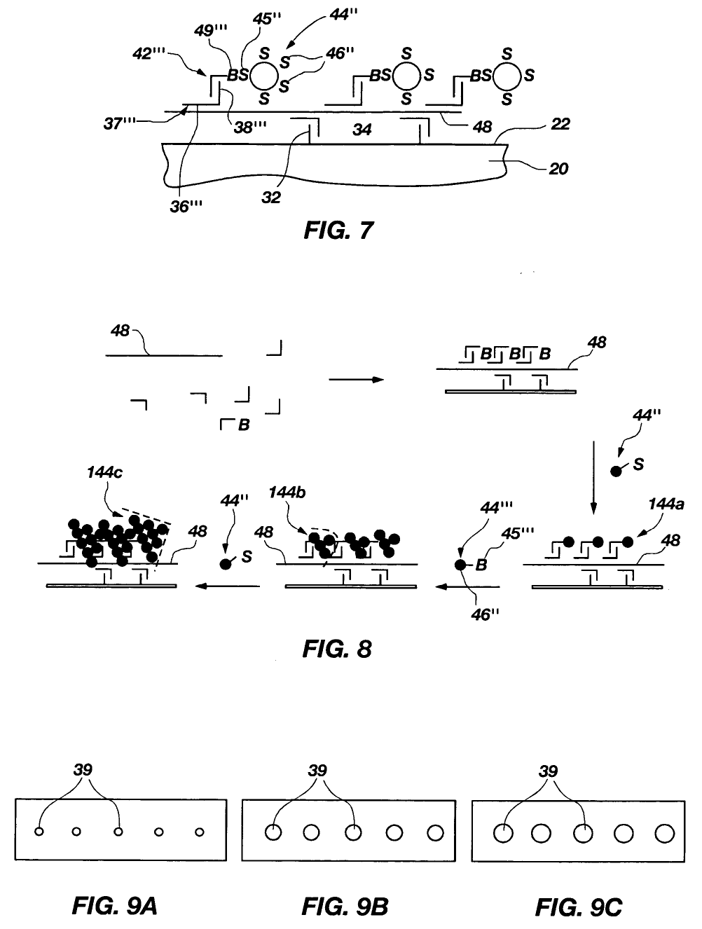

Alternatively, as shown in FIG. 7, a member 49′″ of a binding pair (e.g., biotin-biotin binding protein pair, etc.) may be indirectly bound to a target molecule 48. This embodiment is useful when amplification of target molecule 48 is not necessary, or when unlabeled target molecules 48 are synthesized during amplification.

Member 49′″ binds with a complementary binding pair member 45″ of a signal complex 44″ (see also FIG. 6). As illustrated, each member 49′″ is conjugated to a probe olignucleotide 42′″, which has a nucleotide sequence that is configured to hybridize with a complementary nucleotide sequence of an intermediate, extender oligonucleotide 37′″. Extender oligonucleotide 37′″, in turn, includes a probe oligonucleotide-specific region 38′″ and a target molecule-specific region 36′″. Signal oligonucleotide-specific region 38′″ has a nucleotide sequence that will hybridize with a complementary sequence of nucleotides of a corresponding probe oligonucleotide 42′″. Target molecule-specific region 36′″ has a nucleotide sequence that is configured to hybridize with a complementary sequence of nucleotides along at least a region of target molecule 48.

To illustrate this embodiment, Group A Streptococcus (GAS) DNA (i.e., target molecule 48) was mixed, either individually or concurrently, with GAS-specific bridge oligonuclotides 34 and GAS-specific extender oligonucleotides 37′″, as well as with probe oligonucleotides 42′″. Target molecule 48, which is double stranded in its native state, is exposed to sufficient heat to separate the strands. When target molecule 48 has been denatured, regions of bridge oligonucleotides 34 and extender oligonucleotides 37′″ that are complementary to regions of target molecule 48 may hybridize with their complementary regions (e.g., during incubation or other exposure).

Bridge oligonucleotides 34 hybridize with complementary capture oligonucleotides 32 to immobilize target molecules 48 to surface 22 of waveguide 20, as described above in reference to FIG. 3C.

Additionally, binding pair members 49′″ are exposed to (e.g., incubated with) signal complexes 44″, which, with extender oligonucleotides 37′″, indirectly bind markers 46″ to target molecules 48.

Once target molecules 48 have been immobilized relative to surface 22 of waveguide 20 and signal complexes 44″ have been bound to target molecules 48″, detection may be effected. For example, the fluorescence excitation and detection processes that have been described above in reference to FIG. 5 may be used.

### EXAMPLE 9

The signal generated by the processes described in EXAMPLE 7 and EXAMPLE 8 may be amplified by “cascading techniques,” in which multiple signal complexes 44″ may be indirectly bound to a target molecule 48. An example of such a cascading technique is depicted in FIG. 8.

Without limiting the scope of the present invention, signal complexes 44″ may be bound to target molecules 48 in the same manner that has been described above in reference to FIG. 7 to form a first layer 144a of signal complexes 44″ on target molecule 48.

By way of example, and not to limit the scope of the present invention, once target molecules 48 have been immobilized relative to surface 22 of waveguide 20 and labeled with signal complexes 44″, surface 22 and, thus, target molecules 48 thereover, may be washed, as known in the art, to remove excess bridge oligonucleotides 34″ and signal complexes 44″.

Thereafter, an additional layer 144b of signal complexes 44′″ may be added. Like signal complexes 44″, signal complexes 44′″ include a marker 46″. Rather than including one or more molecules of a biotin binding protein 45″ (FIG. 6), however, signal complexes 44′″ include at least one biotin molecule 45′″ conjugated to each marker 46″. Consequently, when signal complexes 44″ that label a target molecule 48 are exposed to (e.g., incubated with) signal complexes 44′″ (e.g., for a duration of about ten minutes), biotin binding proteins 45″ of signal complexes 44″ bind the biotin molecules 45′″ of signal complexes 44′″.

Additional layers 144c, 144d, etc., which alternately include signal complexes 44″ and 44′″, may also be formed. The formation of additional layers 144b, 144c, etc., follows the same protocol: surface 22 is washed to remove excess signal complex 44″, 44′″, which was used to form the previous layer (e.g., layer 144a, 144b), therefrom, then signal complexes 44′″, 44″ that have been bound to target molecule 48 are exposed to (e.g., incubated with) signal complexes 44″, 44′″that may bind thereto (e.g., for a duration of about ten minutes).

Each additional layer 144b, 144c, 144d, etc., provides for further enhancement of the intensity of a signal that may be generated to indicate the presence or amount of target molecule 48 present in a sample and, thus, may contribute to an increase in the sensitivity of the assay.

As illustrated in FIGS. 9A through 9C, using quantum dot-streptavidin conjugate signal complexes 44″ (FIG. 8) and quantum dot-biotin conjugate signal complexes 44′″ (FIG. 8), this concept has been reduced to practice in detection of Group A Streptococcus DNA. FIG. 9A shows the intensity of the fluorescent signal, at spots 39, generated when a 405 nm laser beam was directed into a waveguide 20 (FIG. 8) to generate an evanescent field over surface 22 to excite a single layer 144a of signal complexes 44″ that had been bound to a target molecule 48 immobilized relative to surface 22. FIG. 9B shows the intensity of the signal, at spots 39, that was generated following the addition of another layer 144b (FIG. 8) of signal complexes 44′″ to target molecule 48. FIG. 9C depicts the intensity of the signal, at spots 39, generated after a third layer 144c (FIG. 8) of signal complexes 44″ was added to target molecule 48.

### EXAMPLE 10

The streptavidin and biotinylated quantum dots may be encapsulated in controlled release capsules, of a type known in the art, that would dissolve in sequence instead of requiring washing between binding steps, as described in EXAMPLE 9. For example, streptavidin-labeled quantum dots (e.g., signal complex 44′″) may be released from time release capsules after several minutes into the reaction time in order to bind to the biotinylated oligonucleotide captured on the surface of the planar waveguide during the initial part of the incubation. After several more minutes, a second controlled release capsule would dissolve, releasing a preformed matrix of streptavidin-labeled and biotin-labeled quantum dots that would bind to the oligonucleotide and the streptavidin quantum dot adduct on the surface of the planar waveguide. This would result in a significant increase in signal intensity at the surface of the planar waveguide. The reaction sequence would be a forward sequential reaction. All reagents would be present in the initial reaction mixture so the reagent formulation would be a homogeneous assay configuration. This reagent encapsulation assay will work with a forward sequential immunoassay as well.

Teachings of the present invention are also applicable to other types of assays, including, without limitation, various types of immunoassays, protein-protein interaction assays (e.g., as used in some phage displays, enzyme-substrate interaction, etc.), and the like.

The following EXAMPLE describes a process for preparing an antibody that may be used in an immunoassay to facilitate binding of a marker to a target molecule.

### EXAMPLE 11

An antibody that is useful as a reagent in an immunoassay for Salmonella typhimurium was prepared using one milligram (1 mg) of affinity-purified goat anti-salmonella CSA-1 antibody, available from Kirkegaard & Perry Laboratories, Inc., of Gaithersburg, Md. The goat anti-salmonella CSA-1 antibody was biotinylated with a ten-fold excess of NHS-LC-LC-Biotin in 0.1 M sodium borate, pH 8.3, for one hour at ambient temperature. The biotinylation reaction was quenched with 50 μL of 0.5 M Tris, pH 8.1, for 15 minutes. Next, the biotinylated goat anti-salmonella CSA-1 antibody was desalted and buffer-exchanged on a CENTRICON® YM-30 centrifugation filter device into 20 mM sodium phosphate, pH 7.2, with 150 mM NaCl and 0.05% sodium azide (PBS).

Thereafter, the biotinylated goat anti-salmonella CSA-1 antibody was hybridized with a signal complex that includes a biotin binding protein-in this case, QDOT® 655 Streptavidin Conjugate. Based on the assumption that there are 20 streptavidin molecules attached to each marker (i.e., quantum dot nanoparticle) of the QDOT® 655 Streptavidin Conjugate signal complex, a 750 pM solution of the QDOT® 655 Streptavidin Conjugate was conjugated to a 20-fold excess of biotinylated antibody to saturate the biotin binding sites on each streptavidin molecule of the signal complex. After incubating for one hour, the antibody-biotin: streptavidin-marker complex was diluted to 0.75 nM in particle units (1.0 nM antibody) in a diluent including 150 mM HEPES, pH 6.1, with 54 mg bovine serum albumin (BSA)/ml and 18 mg sucrose/ml.

The reagents identified in EXAMPLE 11 were used in a waveguide immunoassay, as described in EXAMPLE 12.

### EXAMPLE 12

A BioCentrex cartridge that included a planar waveguide with a surface including a capture phase in the form of spots of 1.5 pmoles of affinity-purified goat anti-salmonella CSA-1 antibody immobilized thereto was used to evaluate binding between the reagents of EXAMPLE 11 and heat-killed Salmonella typhimurium (also from Kirkegaard & Perry Laboratories, Inc.)

One hundred (100) μL of the 0.75 nM solution of the antibody-biotin: streptavidin-marker complex of EXAMPLE 11 was mixed with 200 μL of different concentrations of analyte, in this case heat-killed Salmonella typhimurium (amounting to two tests each of 0 cells/200 μL, 1×10⁶ cells/200 μL, and 1 x 107 cells/200 μL). These mixtures were placed into the reagent cups of different BioCentrex cartridges of the type described in the preceding paragraph.

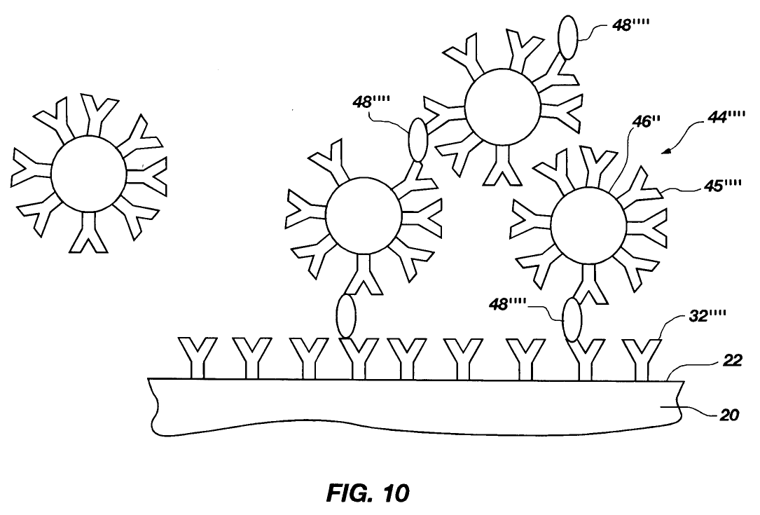

As shown in FIG. 10, when the sample and reagents were incubated with or otherwise exposed to each other, target molecules 48″″ (e.g., heat-killed S. typhimurium) bind with signal complexes 44″″ (e.g., each complex including one or more of the affinity-purified goat anti-salmonella CSA-1 antibody 45″″ molecules complexed to a quantum dot 46″). As shown, one target molecule 48″″ may bind with more than one signal complex 44″″, resulting in something of a cascade effect (i.e., multiple markers 46″ per target molecule 48″″). When the sample-reagent mixture is introduced onto the surface 22 of a waveguide 20, capture molecules 32 (e.g., the affinity-purified goat anti-salmonella CSA-1 antibody), which are immobilized on surface 22, bind to, or “capture,” target molecules 48.

The cartridges were then individually run in BioCentrex analyzers to determine whether or not any signal complex 44″″-labeled target molecules 48″″ had been immobilized relative to surface 22 of waveguide 20.

One cartridge with each concentration of heat-killed Salmonella typhimurium was run in a BioCentrex Analyzer that had been equipped with a 405 nm blue-violet laser, a 530 nm long pass filter, and a CCD camera. As a sandwich immune complex was forming between the capture phase, the analyte, and the reagents, the laser introduced a laser beam into an edge of the planar waveguide and the CCD camera was used to monitor formation of the sandwich immune complex. Such monitoring was effected for a duration of eight minutes, with 0.2 second exposure times that were spaced at 6.5 second intervals.

The other cartridge of each concentration of heat-killed Salmonella typhimurium was run in a BioCentrex Analyzer that had been equipped with a 658 nm red laser, a 703 nm band pass filter, and a CCD camera. As a sandwich immune complex was forming between the capture phase, the analyte, and the reagents, the laser introduced a laser beam into an edge of the planar waveguide and the CCD camera was used to monitor formation of the sandwich immune complex. Such monitoring was effected for a duration of eight minutes, with two second exposure times that were spaced at 6.5 second intervals.

The mean fluorescent rates are presented in TABLE 3.

TABLE 3

Mean Fluorescent Rate
Test

| Cells | 405 nm | 658 nm |
|---|---|---|
| 0 | 0.75 | 0.5 |
| 1,000,000 | 1.5 | 0.75 |
| 10,000,000 | 4.5 | 2.5 |

Although the EXAMPLES describe sandwich-type assays, other types of assays, including so-called “competition assays,” in which marker-labeled molecules compete with analyte molecules for binding sites on capture molecules, are also within the scope of the present invention.

Based on experiments that have been conducted, as set forth in some of the preceding EXAMPLES, it is believed that quantum dot-labeled gene-specific oligonucleotide probes provide orders of magnitude higher fluorescence than that provided by oligonucleotide probes that have been labeled with organic fluorescent molecules, such as CY3 and CY5. Therefore, it is also believed that quantum dot-based assays provide orders of magnitude higher sensitivity than assays that employ traditional organic fluorescent molecules.

Although the foregoing description contains many specifics, these should not be construed as limiting the scope of the present invention, but merely as providing illustrations of some of the presently preferred embodiments. Similarly, other embodiments may be devised without departing from the spirit or scope of the present invention. Features from different embodiments may be employed in combination. The scope of the invention is, therefore, indicated and limited only by the appended claims and their legal equivalents rather than by the foregoing description. All additions, deletions and modifications to the invention as disclosed herein which fall within the meaning and scope of the claims are to be embraced thereby.

1. An assay system, comprising:
a waveguide;
at least one type of capture molecule carried upon at least one surface of the waveguide for selectively binding at least one species of target molecule or a corresponding competitive molecule;
a reagent solution including quantum dots for indicating a presence or an amount of the at least one target molecule in a sample.

2. The assay system of claim 1, further comprising:
an excitation source configured to direct at least one wavelength of excitation radiation into the waveguide to excite quantum dots located within an evanescent field over the at least one surface of the waveguide; and
a detector configured to sense at least one wavelength of emission radiation fluoresced by the quantum dots.

3. The assay system of claim 2, wherein the excitation source is oriented to direct the radiation into the waveguide in such a way that the radiation will be internally reflected within the waveguide.

4. The assay system of claim 2, wherein the excitation source emits radiation having a wavelength of about 800 nm or less.

5. The assay system of claim 1, wherein the at least one type of quantum dots exhibits a Stoke's shift of about 50 nm or greater.

6. The assay system of claim 1, wherein the at least one type of quantum dots exhibits a Stoke's shift of about 100 nm or greater.

7. The assay system of claim 1, wherein the waveguide comprises a planar waveguide, a cylindrical waveguide, or a spherical waveguide.

8. The assay system of claim 1, wherein the waveguide comprises a thin film waveguide.

9. The assay system of claim 1, wherein the at least one analyte comprises a nucleic acid, an antigen, or an antibody.

10. The assay system of claim 1, wherein the reagent solution is configured to effect a sandwich-type assay.

11. The assay system of claim 10, wherein the reagent solution comprises signal complexes that are configured to bind the target molecule and that include the quantum dots.

12. The assay system of claim 1, wherein the reagent solution is configured to effect a competition-type assay.

13. The assay system of claim 12, wherein the reagent solution comprises the competitive molecules, which include the quantum dots.

14. The assay system of claim 1, wherein the quantum dots exhibit a Stoke's shift of about 50 nm or greater.

15. The assay system of claim 1, wherein the quantum dots exhibit a Stoke's shift of about 100 nm or greater.

16. A method for conducting an assay, comprising:
exposing a sample to a reagent solution that includes at least one type of signal complex for labeling at least one species of target molecule in the sample;
exposing the at least one type of signal complex to additional signal complex including a marker of the same type to form an additional layer of signal complex; and
selectively binding at least one of the at least one species of target molecule and a corresponding species of competitive molecule to a solid phase.

17. The method of claim 16, wherein exposing the sample to the reagent solution comprises exposing the sample to a reagent solution with the at least one type of signal complex including a marker with at least one first binding pair member conjugated thereto.

18. The method of claim 17, wherein exposing the at least one type of signal complex to additional signal complex comprises exposing the at least one type of signal complex to additional signal complex with at least one second binding pair member conjugated to the marker thereof.

19. The method of claim 18, wherein exposing the at least one type of signal complex to additional signal complex comprises exposing a signal complex including one of biotin and a biotin binding protein to additional signal complex including sample to the other of biotin and a biotin binding protein.

20. The method of claim 16, wherein exposing the sample to the reagent solution includes exposing the sample to a reagent solution with the at least one type of signal complex including a marker that comprises a quantum dot.

21. The method of claim 20, wherein exposing the at least one type of signal complex to additional signal complex includes exposing the at least one type of signal complex to additional signal complex with the marker thereof comprising a quantum dot.

22. The method of claim 16, further comprising:
exposing the additional signal complex to more additional signal complex that will bind thereto to form at least one additional layer of signal complex.

23. The method of claim 16, wherein selectively binding comprises selectively binding at least one species of target molecule or the corresponding species of competitive molecule to capture molecules immobilized to a surface of a waveguide.

24. The method of claim 23, further comprising:
directing electromagnetic radiation into the waveguide to generate an evanescent field at a surface thereof, markers of the at least one type of signal complex and the additional signal complex within the evanescent field being excited by the evanescent field; and
detecting excitation of the markers.

25. The method of claim 24, wherein:
directing electromagnetic radiation comprises directing electromagnetic radiation into the waveguide to generate the evanescent field to cause the markers to fluoresce emission radiation; and
detecting excitation comprises detecting the emission radiation.

26. The method of claim 25, wherein directing electromagnetic radiation comprises directing electromagnetic radiation having a wavelength of about 800 nm or less into the waveguide.

27. The method of claim 25, wherein detecting excitation comprises detecting emission radiation having a wavelength of at least about 50 nm greater than an excitation wavelength of radiation directed into the waveguide.

28. The method of claim 25, wherein detecting excitation comprises detecting emission radiation having a wavelength of at least about 100 nm greater than an excitation wavelength of radiation directed into the waveguide.

29. A method for effecting a biological assay, comprising:
exposing a sample solution potentially including at least one species of target molecule to a reagent solution including quantum dots having a Stoke's shift of about 50 nm or greater for indicating a presence or an amount of the at least one target molecule in the sample;
introducing the sample solution onto the surface of a waveguide to selectively bind the at least one species of target molecule or a corresponding species of competitive molecule to capture molecules that have been immobilized to the surface;
directing, into the waveguide, excitation radiation having a wavelength that will generate an evanescent field at a surface of the waveguide that will excite quantum dots that have been immobilized relative to the surface;
detecting at least one wavelength of emission radiation fluoresced by the quantum dots; and
correlating the at least one wavelength of emission radiation to a presence or amount of the at least one species of target molecule present in the sample.

30. The method of claim 29, wherein directing comprises directing excitation radiation having a wavelength of about 800 nm or less into the waveguide.

31. The method of claim 30, wherein detecting comprises detecting emission radiation having a wavelength of at least about 500 nm.

32. The method of claim 29, wherein detecting comprises detecting at least one wavelength of emission radiation that is about 50 nm or greater than the wavelength of the excitation radiation.

33. The method of claim 29, wherein detecting comprises detecting at least one wavelength of emission radiation that is at least about 100 nm greater than the wavelength of the excitation radiation.

34. The method of claim 29, wherein exposing includes binding a plurality of signal complexes including the quantum dots to at least one target molecule.

35. The method of claim 34, wherein binding comprises cascading additional signal complexes to a signal complex that has been bound to the at least one target molecule.
